(19) United States
(12) Patent Application Publication
(10) Pub. No.: US 2007/0160272 A1
(43) Pub. Date: Jul. 12, 2007

(54) Tumor region setting method and system

(76) Inventors: Yoshihiko Nagamine, Hitachi (JP); Takuro Honda, Mito (JP)

Correspondence Address: DICKSTEIN SHAPIRO LLP, 1825 EYE STREET NW, Washington, DC, 20006-5403, US

(21) Appl. No.: 11/327,345

(22) Filed: Jan. 9, 2006

(51) Int. Cl. G06K 9/00; A61B 5/05

(52) U.S. Cl. 382/128; 600/410

(57) ABSTRACT

A tumor region setting method and system capable of simply and accurately setting a tumor region. A processor extracts contour data of a tumor region from a PET image read out of a medical image server and converts the extracted contour data to vector line data. A monitor displays the tumor region represented by the vector line data in superimposed relation to an X-ray CT image read out of the medical image server.

### BACKGROUND OF THE INVENTION

1. Field of the Invention

The present invention relates to a tumor region setting method and system used in radiation medicine for setting a tumor region based on nuclear medicine image information that is obtained from a nuclear medicine diagnosis apparatus.

2. Description of the Related Art

Radiation treatment is generally progressed through successive steps, i.e., (1) capturing of an X-ray CT image of a patient, (2) planning of a treatment plan based on the X-ray CT image, and (3) treatment using radiation irradiation equipment in accordance with the treatment plan. Main purposes of the treatment plan are to set a tumor region on the X-ray CT image and to set a dose irradiated to the set tumor region. The tumor region is set, for example, by a direct setting method on the X-ray CT image, or a method combined with an MRI image (i.e., an image captured by an MRI (Magnetic Resonance Imaging) system) that is superior in observing soft tissues to the X-ray CT image.

Recently, attention has been focused on a positron CT (PET: Positron Emission Tomography) system, as radiation diagnosis equipment useful for finding malignant tumors at earlier timing, in Japan and many other countries. The PET system is radiation diagnosis equipment in which a radionuclide released upon a positron decay is applied as a drug into a human body and a pair of γ-rays (511 KeV) released upon pair annihilation of a positron and an electron occurred in the body are captured for imaging. The drug is provided as, e.g., FDG (2-deoxy-2-fluoro-D-glucose) that is a similitude of glucose. Because FDG tends to accumulate on a tumor showing vigorous energy metabolism, the tumor can be imaged by utilizing such a tendency.

Unlike an anatomical image representing the shapes of a body and organs, such as the X-ray CT image and the MRI image, the image captured by the PET system is an image reflecting the activity of the cell function and representing the tumor region (so-called functional image). By using the PET image in setting of the tumor region in combination with the X-ray CT image and the MRI image, therefore, it becomes possible to set the tumor region that is difficult to find with the X-ray CT image and the MRI image.

As the related art employing the PET image in a combined manner as described above, there are known techniques of displaying the X-ray CT image and the PET image in superimposed relation with proper alignment, and setting the tumor region based on the superimposed image (see Patent Document 1; JP,A 9-133771 and Patent Document 2; JP,A 2000-105279).

### SUMMARY OF THE INVENTION

Though not clearly explained in Patent Documents 1 and 2, a medical image, such as an X-ray CT image and a PET image, is generally constituted as raster data (two-dimensional array in the form of pixel sets), and an image obtained by superimposing the PET image on the X-ray CT image is also constituted as raster data. On the other hand, data used in a treatment plan for defining the tumor region is usually prepared in the format using vector line data (i.e., lines each having a magnitude and a direction and points), such as in a polygon line drawing. The above-mentioned related art, therefore, accompanies problems given below. Even with the PET image displayed in superimposed relation to the X-ray CT image, an operator cannot employ the tumor region, which can be discerned with data of the PET image, directly as a tumor region for the treatment plan. Stated another way, the operator has to manually trace the tumor region, which can be discerned with the data of the PET image, by using an input unit, e.g., a mouse, for conversion to vector line data. Such a manual process requires time and labor in setting the tumor region, and inevitably causes differences in tracing results of the tumor region depending on the skills and other factors of individual operators, thus resulting in lower accuracy in definition of the tumor region.

In view of the above-mentioned problems with the related art, an object of the present invention is to provide a tumor region setting method and system capable of simply and accurately setting a tumor region.

(1) To achieve the above object, according to the present invention (first form corresponding to claim 1), in a tumor region setting method for use in radiation treatment, the method comprises the steps of extracting contour information of a tumor region from nuclear medicine image information; and converting the extracted contour information to vector line information.

In the present invention, when a threshold is set in terms of a count value or a SUV value, the contour of the tumor region is extracted from a PET image and contour data is converted to vector line data by a processor in an automatic manner. It is therefore possible to eliminate the necessity of tracing the contour of the tumor region by the operator's hand unlike the related art, and to noticeably simplify the operation for setting the tumor region. Further, since the contour is automatically extracted based on the threshold, the tumor region can be set with high reproducibility regardless of the skills and other factors of individual operators. As a result, the present invention enables the tumor region to be simply and accurately set.

(2) According to a second form of the present invention, in the tumor region setting method of above (1), the contour information of the tumor region is extracted from the nuclear medicine image information by using a threshold.

(3) According to a third form of the present invention, in the tumor region setting method of above (2), the threshold is set in terms of a radioactivity count value contained in the nuclear medicine image information.

(4) According to a fourth form of the present invention, in the tumor region setting method of above (2), the threshold is set in terms of a SUV value.

(5) According to a fifth form of the present invention, in the tumor region setting method of above (2), the vector line information and X-ray CT image information are displayed in superimposed relation.

(6) According to a sixth form of the present invention, in the tumor region setting method of above (5), the vector line information is Bezier curve information.

(7) According to a seventh form of the present invention, in the tumor region setting method of above (5), the vector line information is polygon line information.

(8) According to an eighth form of the present invention, in the tumor region setting method of above (5), the nuclear-medicine image information is PET image information.

(9) To achieve the above object, according to a ninth form of the present invention, in a tumor region setting system for setting a tumor region in radiation treatment, the system comprises an image server for storing X-ray CT image information and nuclear medicine image information each including a tumor region; a processor for extracting contour information of a tumor region from the nuclear medicine image information and converting the extracted contour information to vector line information; and a display for displaying the vector line information and the X-ray CT image information in superimposed relation.

(10) To achieve the above object, according to a tenth form of the present invention, in a tumor region setting system for setting a tumor region in radiation treatment, the system comprises an image server for storing X-ray CT image information and nuclear medicine image information each including a tumor region; a processor for extracting contour information of a tumor region from the nuclear medicine image information and converting the extracted contour information to vector line information; and a display for displaying side by side first image information given by the X-ray CT image information and second image information obtained by superimposing the X-ray CT image information and the nuclear medicine image information, and displaying the vector line information converted by the processor in superimposed relation to the first image information.

(11) To achieve the above object, according to an eleventh form of the present invention, in a tumor region setting system for setting a tumor region in radiation treatment, the system comprises an image server for storing X-ray CT image information, nuclear medicine image information, and MRI image information each including a tumor region; a processor for extracting contour information of a tumor region from the nuclear medicine image information and converting the extracted contour information to vector line information; and a display for displaying side by side first image information given by the X-ray CT image information and second image information obtained by superimposing the MRI image information and the nuclear medicine image information, and displaying the vector line information converted by the processor in superimposed relation to the first image information.

According to the present invention, the tumor region can be simply and accurately set by extracting the contour information of the tumor region from the nuclear medicine image information and converting the extracted contour information to the vector line information.

### BRIEF DESCRIPTION OF THE DRAWINGS

FIG. 1 is a schematic view showing the entire configuration of a tumor region setting system according to one embodiment of the present invention;

FIG. 2 is a flowchart showing a process flow executed by the tumor region setting system shown in FIG. 1;

FIGS. 3A and 3B show examples of images displayed on a monitor shown in FIG. 1; specifically, FIG. 3A shows one example in which an X-ray CT image and an image obtained by superimposing a PET image over the X-ray CT image are displayed side by side, and FIG. 3B shows the other example in which an image obtained by superimposing vector line data, which represents the contour of a tumor region, over the PET image and the image obtained by superimposing the PET image over the X-ray CT image are displayed side by side;

FIG. 4 is a representation showing a threshold setting dialog box;

FIGS. 5A-5D are illustrations for explaining a sampling process for the contour data of the tumor region;

FIG. 6 is an illustration for explaining a Bezier curve; and

FIG. 7 is an illustration for explaining a process for converting the contour data to a Bezier curve.

### DESCRIPTION OF THE PREFERRED EMBODIMENTS

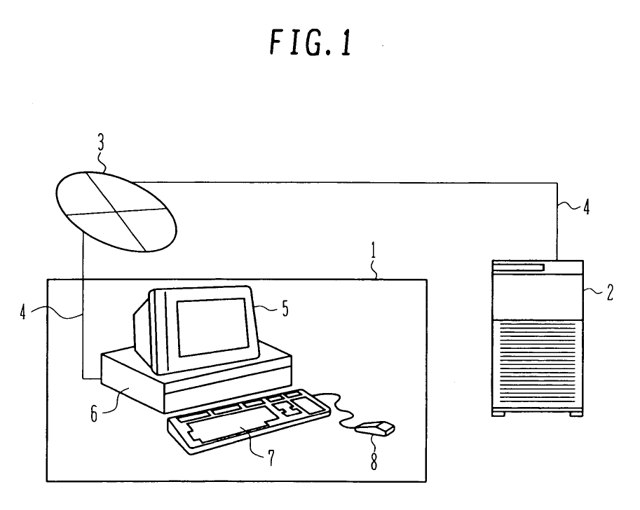

FIG. 1 is a schematic view showing the entire configuration of a tumor region setting system according to one embodiment of the present invention. In FIG. 1, the tumor region setting system comprises a treatment planning unit 1 and a medical image server 2 which archives an X-ray CT image, a PET image (nuclear medicine image information), etc. The treatment planning unit 1 and the medical image server 2 are connected to, e.g., an intra-hospital network 3, which is laid in a hospital, via a network cable 4. The treatment planning unit 1 comprises a monitor (display) 5 for displaying a medical image and medical information, a processor 6, a keyboard 7, and a mouse 8 serving as an input unit. The mouse 8 may be replaced with any other suitable input unit, such as a pen tablet.

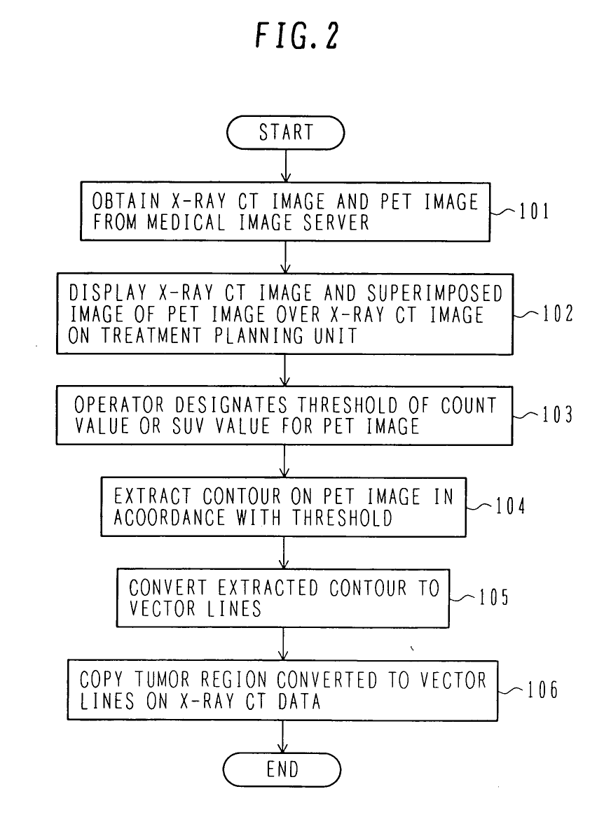

FIG. 2 shows a processing flow executed by the tumor region setting system of the embodiment. A tumor region setting method according to the present invention will be described below with reference to FIG. 2.

An operator using the treatment planning unit 1 first obtains an X-ray CT image and a PET image for a treatment plan of a patient, for whom the treatment plan is going to be planned, from the medical image server 2 (step 101).

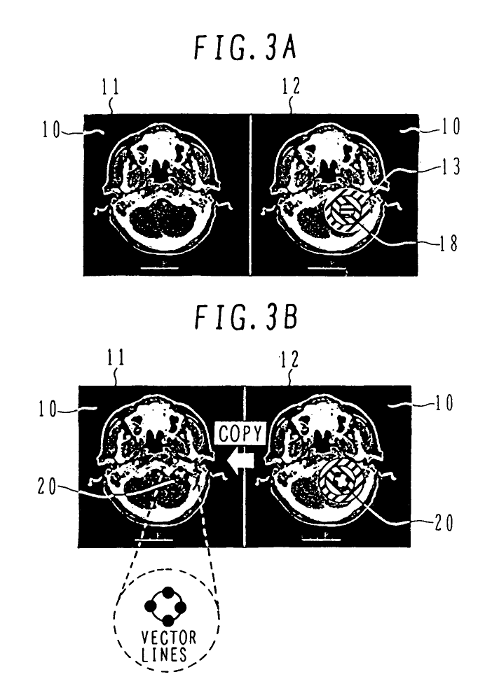

After the medical images have been obtained from the medical image server 2, the X-ray CT image (first image information) and a superimposed image (second image information) of a PET image over the X-ray CT image are displayed side by side on the monitor 5 of the treatment planning unit 1 (step 102). The state of the monitor 5 at this time is as shown in FIG. 3A. For example, only an X-ray CT image 10 is displayed in an image display area 11 on the left side, and an image obtained by superimposing a PET image display area 13 (also called a “PET image 13” hereinafter) over the X-ray CT image 10 is displayed in an image display area 12 on the right side.

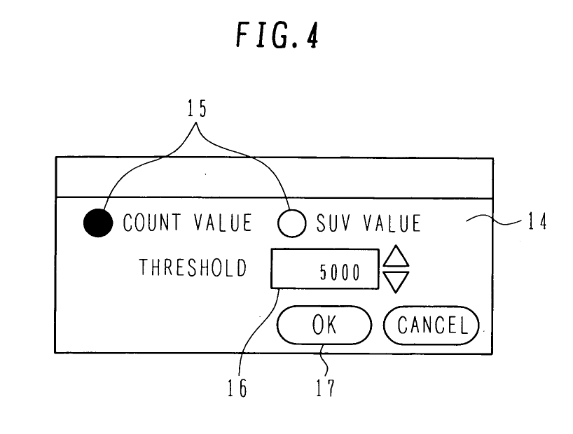

After the images are displayed, the operator designates a threshold used for extracting a contour with respect to the PET image display area 13 (step 103). The threshold can be designated in terms of either a radioactivity count value or a SUV value. Here, the term “radioactivity count value” means a radioactivity count value (referred to as a “count value” hereinafter) that is originally stored as raster data of the PET image. The radioactivity count value is given as a value detected by a radiation detector associated with a PET system. On the other hand, the term “SUV (Standardized Uptake Value)” means a value resulting from dividing the count value by an average count value obtained in the state where the drug is uniformly distributed in a patient body. SUV=1 represents an average distribution of the drug. The SUV value in excess of 2 to 3, for example, indicates a possibility of the presence of a tumor. If the PET image is displayed in terms of the count value when the operator is going to designate the threshold in terms of the SUV value, the PET image is switched to be displayed in terms of the count value upon selection of the threshold. Conversely, if the PET image is displayed in terms of the SUV value when the operator is going to designate the threshold in terms of the count value, the PET image is switched to be displayed in terms of the count value. Setting of the threshold is made using a threshold setting dialog box 14 shown in FIG. 4. The operator clicks one of radio buttons 15 for selecting the type of the threshold and enters a value in a text field 16 where the threshold is to be inputted. Then, the operator pushes an OK button 17, whereby the setting of the threshold is completed. In the embodiment, it is assumed that a conversion area 18 within the PET image 13, shown in FIG. 3A, is designated as a tumor region for which the threshold is set for extraction.

After the setting of the threshold, the processor 6 automatically extracts the contour of the tumor region on the PET image 13 (step 104). The PET image is a raster image, and the contour extraction for the raster image can be performed by using a generally known algorithm (see, e.g., “Practical Image Processing Learned with C Language”, Ohmsha (Japan), 1999). More specifically, pixels exceeding the threshold are sought starting from a pixel at an upper left corner of the PET image, to thereby form an image made up of only the pixels exceeding the threshold. On that occasion, the pixels exceeding the threshold are all converted to the same value (thus-converted image data is called a binary image). The contour of the tumor region demarcated depending on the threshold can be formed on the PET image 13 by applying a known edge extraction algorithm, e.g., the Pewitt's method, to the binary image. Further, the extracted contour is converted to a contour having a width of one pixel by using a known thin line algorithm, e.g., the Hildrich's method.

After the contour extraction on the PET image 13, the processor 16 converts the contour data, which has been automatically extracted and converted to a thin line having a width of one pixel, to vector line data (step 105). The term “vector line” means a line having a magnitude and a direction, such as a Bezier curve or a polygon line, and the term “vector line data (drawing)” means a line drawing made up of a train of points and lines interconnecting the train of points. Procedures and manners for converting the contour data to the vector line (Bezier curve in the embodiment) data will be described below.

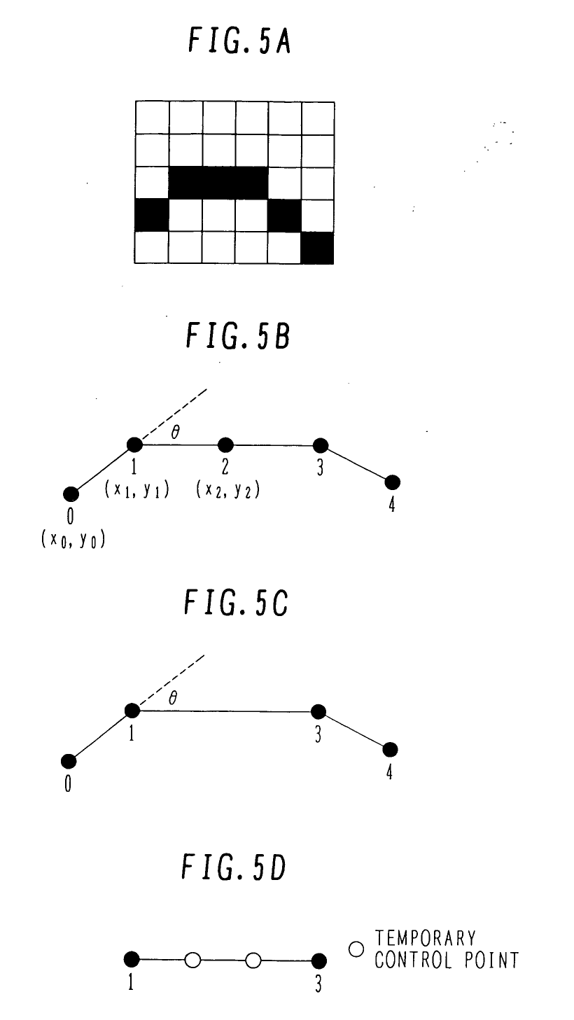

First, the contour data is obtained in the form where a line having a width of one pixel is continued as shown in FIG. 5A. Note that, in FIG. 5A, a part of the contour data is shown in enlarged scale for the purpose of explanation. If the contour data is directly converted to the vector line data, useless operations are increased and therefore the points are thinned by sampling. The sampling is performed such that the original shape of the contour data is not lost. In the embodiment, the sampling is performed based on the curvature between the pixels. In other words, the pixels having the curvatures not larger than a certain value are omitted. To explain in more detail, numbers are allocated to the pixels shown in FIG. 5A. FIG. 5B schematically shows the numbered pixels. The numbered pixels are assumed to have coordinates (x0, y0), (x1, y1) and (x2, y2). Looking from No. 1 point, an angle θ formed by a line connecting Nos. 0 and 1 points and a line connecting Nos. 1 and 2 points is expressed by the following formula 1 on an assumption that cosine of θ is defined as a curvature R:

$$\begin{matrix}R = cos θ \\ = \frac{( x 1 - x 0 ) ( x 2 - x 1 ) + ( y 1 - y 0 ) ( y 2 - y 1 )}{\sqrt{{( x 1 - x 0 )}^{2} + {( y 1 - y 0 )}^{2}} \sqrt{{( x 2 - x 1 )}^{2} + {( y 2 - y 1 )}^{2}}}\end{matrix} \qquad ( Formula 1 )$$

Each point having a curvature not larger than the curvature R is successively omitted from the contour data. As a result, data shown in FIG. 5C is obtained. Here, No. 2 point is omitted.

After the contour data has been all scanned in such a way, two points are newly inserted on a linear line between the numbered two points, as shown in FIG. 5D, such that the new points divide the linear line into three equal parts. Those new points are called temporal control points. Then, a Bezier curve is created using the temporal control points and the both-end points sandwiching the formers.

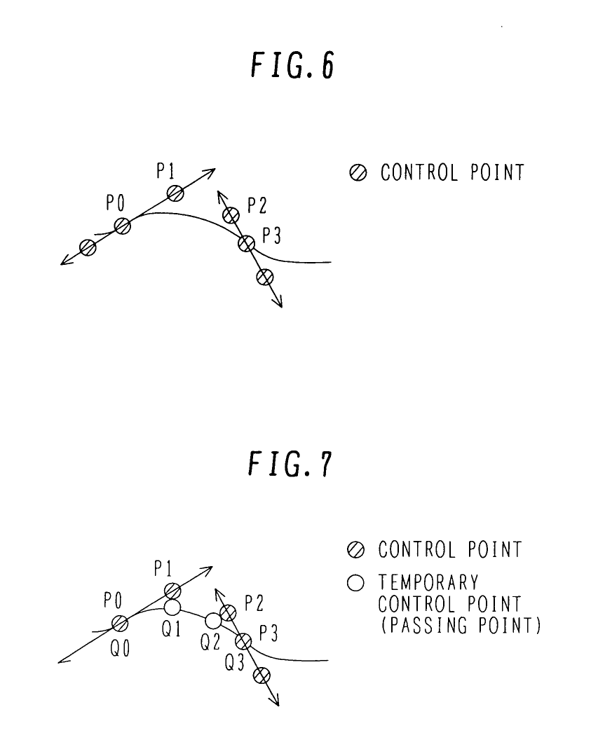

Prior to explaining the conversion of the contour data to the vector line data using the Bezier curve, the term “Bezier curve” will be briefly described. The Bezier curve can be expressed by the following formulae 2 and 3. In the formulae 2 and 3, R(t) represents a curve expressed using a parameter t, and Pi represents a control point. Also, t ranges from 0 to 1, and Bi(t) represents the Bernstein function. A curve shown in FIG. 6 can be created from the formulae 2 and 3. In FIG. 6, P0 to P3 are each a control point, and a curve spanning over P0 to P3 is the Bezier curve.

$$\begin{matrix}R ( t ) = {\sum }_{i = 0}^{3} B {i}^{3} ( t ) Pi & ( Formula 2 ) \\ {Bi}^{3} ( t ) = ( \begin{matrix}3 \\ i\end{matrix} ) {( 1 - t )}^{3 - i} {t}^{i} & ( Formula 3 )\end{matrix}$$

The conversion of the contour data to the vector line data using the Bezier curve is performed as follows. When the contour data after the sampling is approximated by the Bezier curve, four points shown in FIG. 5D are here assumed to be a target of the approximation. The basic concept of the conversion is illustrated in FIG. 7. The target four points are denoted by Q0 to Q3. The Bezier curve has to pass those four points. Also, the Bezier curve has control points which are generally expressed by P0 to P3 as shown in FIG. 7. In other words, the control points P0 to P3 are decided so that the Bezier curve passes the target points Q0 to Q3.

First, P0=Q0 and P3=Q3 hold as seen from FIG. 7. Also, as seen from the formula 2, a curve can be expressed by the parameter t. It is then assumed that the parameter between Q0 and Q1 is t1, the parameter between Q1 and Q2 is t2, and the parameter between Q2 and Q3 is t3. P1 and P2 can be decided using the formula 2 with those parameters. Assuming Q1 and Q2 to be temporal control points, Q1 and Q2 are expressed by the following formulae (4) and (5):

$$\begin{matrix}Q 1 = {\sum }_{i = 0}^{3} {Bi}^{3} ( t 1 ) Pi & ( Formula 4 ) \\ Q 2 = {\sum }_{i = 0}^{3} {Bi}^{3} ( t 2 ) Pi & ( Formula 5 )\end{matrix}$$

By solving the formulae 4 and 5 for P1 and P2, the control points are all decided and the points Q0 to Q3 in FIG. 7 can be approximated by the Bezier curve. Through the procedures described above, the contour data is converted to the vector line data.

After the conversion of the contour data to the vector line data, the processor 6 displays the result on the monitor 5. FIG. 3B shows the state where the result is displayed. In the image display area 12 on the right side, a tumor region 20 converted to the vector line data is displayed in superimposed relation to the superimposed image of the PET image 13 over the X-ray CT image 10. At the same time, the tumor region 20 converted to the vector line data is copied to the X-ray CT image 10 displayed in the image display area 11 on the left side (step 106). Because the tumor region 20 converted to the vector line data is made up of curves each connecting points, the operator is able to edit the tumor region 20 in the form of the vector line data so as to define a more accurate tumor region. The edited result is reflected at once on the X-ray CT image.

After the setting of the tumor region 20 on the X-ray CT image has been completed in such a way, the operator makes further setting of a dose irradiated to the thus-set tumor region, etc.

The tumor region setting method and system thus constructed can provide operating advantages given below.

As mentioned above, in general, a medical image is constituted as raster data (two-dimensional array in the form of pixel sets), while data used in a treatment plan for defining the tumor region is prepared in the format using vector line data (i.e., lines each having a magnitude and a direction and points), such as in a polygon line drawing. In the above-mentioned related art disclosed in Patent Documents 1 and 2, therefore, the following problems arise though not clearly explained in those documents. Even with the PET image displayed in superimposed relation to the X-ray CT image, the operator cannot usually employ the tumor region, which can be discerned with data of the PET image, directly as a tumor region for the treatment plan. Stated another way, the operator has to manually trace the tumor region, which can be discerned with the data of the PET image, by using an input unit, e.g., a mouse, for conversion to polygon line data. Such a manual process requires time and labor in setting the tumor region, and inevitably causes differences in tracing results of the tumor region depending on the skills and other factors of individual operators, thus resulting in lower accuracy in definition of the tumor region.

In contrast, according to the embodiment, when the operator makes just an operation of setting the threshold in terms of the count value or the SUV value, the processor 6 automatically extracts the contour of the tumor region from the PET image 13 and converts the contour data to the vector line data for use in the treatment plan. It is therefore possible to eliminate the necessity of tracing the contour of the tumor region by the operator's hand unlike the related art, and to noticeably simplify the operation for setting the tumor region. Further, since the contour is automatically extracted based on the threshold, the tumor region can be set with high reproducibility (i.e., high reliability) regardless of the skills and other factors of individual operators. As a result, the embodiment enables the tumor region to be simply and accurately set.

While the embodiment has been described above, by way of example, in connection with the case of a vector line being the Bezier curve, it is needles to say that any other suitable vector line, such as a polygon line, may also be used instead of the Bezier curve.

Also, while in the embodiment the X-ray CT image 10 and the PET image 13 are displayed in superimposed relation in the image display area 12 on the right side of the monitor 5, an MRI image is often more suitable for observation depending on a region (e.g., region of soft tissues) for which the treatment plan is going to be planned. In such a case, the MRI image and the PET image 13 may be displayed in superimposed relation in the image display area 12.

Further, while in the embodiment the display screen of the monitor 5 is divided into two parts, i.e., the image display area 11 and the image display area 12, such that the X-ray CT image 10 and the superimposed image of the PET image 13 over the X-ray CT image 10 are displayed side by side in those display areas, the present invention is not limited to such a screen layout. For example, the display screen of the monitor 5 may be provided as one image display area, i.e., only the image display area 12 in which the X-ray CT image 10 and the PET image 13 are displayed in superimposed relation.

What is claimed is:

1. A tumor region setting method for use in radiation treatment, the method comprising the steps of:
extracting contour information of a tumor region from nuclear medicine image information; and
converting the extracted contour information to vector line information.

2. The tumor region setting method according to claim 1, wherein the contour information of the tumor region is extracted from the nuclear medicine image information by using a threshold.

3. The tumor region setting method according to claim 2, wherein the threshold is set in terms of a radioactivity count value contained in the nuclear medicine image information.

4. The tumor region setting method according to claim 2, wherein the threshold is set in terms of a SUV value.

5. The tumor region setting method according to claim 2, wherein the vector line information and X-ray CT image information are displayed in superimposed relation.

6. The tumor region setting method according to claim 5, wherein the vector line information is Bezier curve information.

7. The tumor region setting method according to claim 5, wherein the vector line information is polygon line information.

8. The tumor region setting method according to claim 5, wherein the nuclear medicine image information is PET image information.

9. A tumor region setting system for setting a tumor region in radiation treatment, the system comprising:
an image server for storing X-ray CT image information and nuclear medicine image information each including a tumor region;
a processor for extracting contour information of a tumor region from the nuclear medicine image information and converting the extracted contour information to vector line information; and
a display for displaying the vector line information and the X-ray CT image information in superimposed relation.

10. A tumor region setting system for setting a tumor region in radiation treatment, the system comprising:
an image server for storing X-ray CT image information and nuclear medicine image information each including a tumor region;
a processor for extracting contour information of a tumor region from the nuclear medicine image information and converting the extracted contour information to vector line information; and
a display for displaying side by side first image information given by the X-ray CT image information and second image information obtained by superimposing the X-ray CT image information and the nuclear medicine image information, and displaying the vector line information converted by said processor in superimposed relation to the first image information.

11. A tumor region setting system for setting a tumor region in radiation treatment, the system comprising:
an image server for storing X-ray CT image information, nuclear medicine image information, and MRI image information each including a tumor region;
a processor for extracting contour information of a tumor region from the nuclear medicine image information and converting the extracted contour information to vector line information; and
a display for displaying side by side first image information given by the X-ray CT image information and second image information obtained by superimposing the MRI image information and the nuclear medicine image information, and displaying the vector line information converted by said processor in superimposed relation to the first image information.
